(19) United States
(12) Patent Application Publication
(10) Pub. No.: US 2007/0017514 A1
(43) Pub. Date: Jan. 25, 2007

(54) A Digital gas flowmeter

(76) Inventors: Yang Zheng, Beijing (CN); Timothy Michael Wilkerson, Beijing (CN); Shu Zheng, Beijing (CN)

Correspondence Address: Zheng Yang, 25-1-502,LongYueYuan1,Changping District, Beijing, 102208, CN

(21) Appl. No.: 11/161,006

(22) Filed: Jul. 19, 2005

(51) Int. Cl. A61M 16/00; A62B 7/00

(52) U.S. Cl. 128/204.23

(57) ABSTRACT

A digital gas flowmeter is described as follows: the flowmeter can measure gas flow by digital signal, a optical or magnetic sensor can sense the rotation of a wheel rotating by the pressure of gas that enters into a gas chamber built in the flowmeter; the measure mode can be programmable in on-chip CPU; the digital gas flowmeter includes a gas chamber, which formed by shell, wheel, rotor sensor, gas-in sensor, cone shaped gas-in tube and gas-out tube. The rotor sensor connects with a programmable on-chip CPU, with the rotor sensor recognizes the rotation mechanical signal of the wheel in gas chamber, and converts mechanical signal to digital signal and sends to CPU; this signal can be processed in certain modes programmed by CPU and CPU sends the final result data of gas flow to a display device showing gas usage.

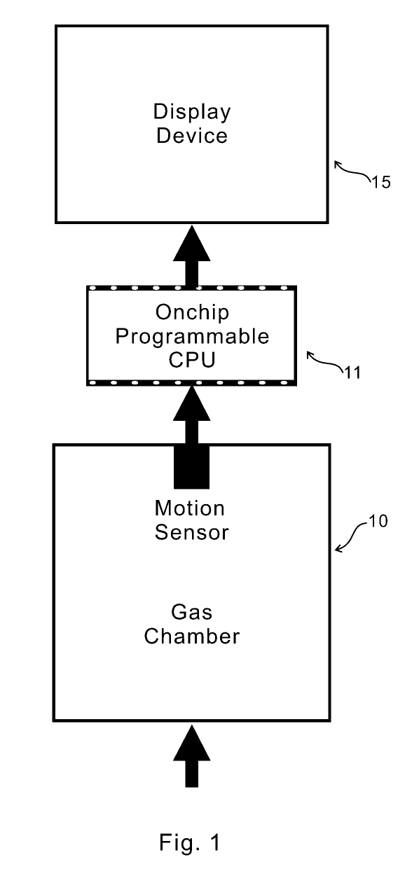

This device measures gas usage such as oxygen or hydrogen through the use of a rotating wheel which will convert the wheel rotation into a pulse signal, then to a digital signal, which is then processed by a CPU which then measures gas usage [FIG. 1].

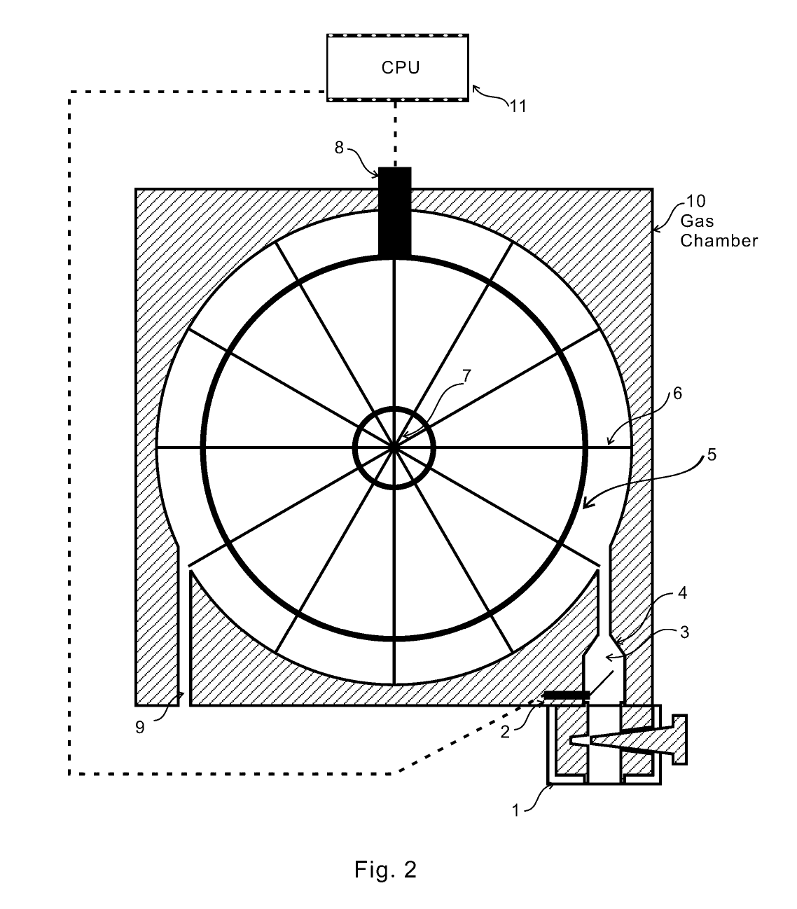

Upon rotation of a mechanical valve 1 [FIG. 2], which has a 360 degree rotation allowing gas flow from 0% to 100%, by user, gas originating from a source such as an oxygen pressurized tank, enters device.

A gas-in sensor 2 [FIG. 2] recognizes the influx of gas. A start signal is sent by sensor to the CPU 11 [FIG. 2] contained within the device allowing CPU 11 to begin calculating gas usage.

Gas enters a tube 3 [FIG. 2] with a cone 4 [FIG. 2] on the exiting side of the tube. The cone has an enter-exit ratio of 15:1. This cone allows for an increase in gas pressure.

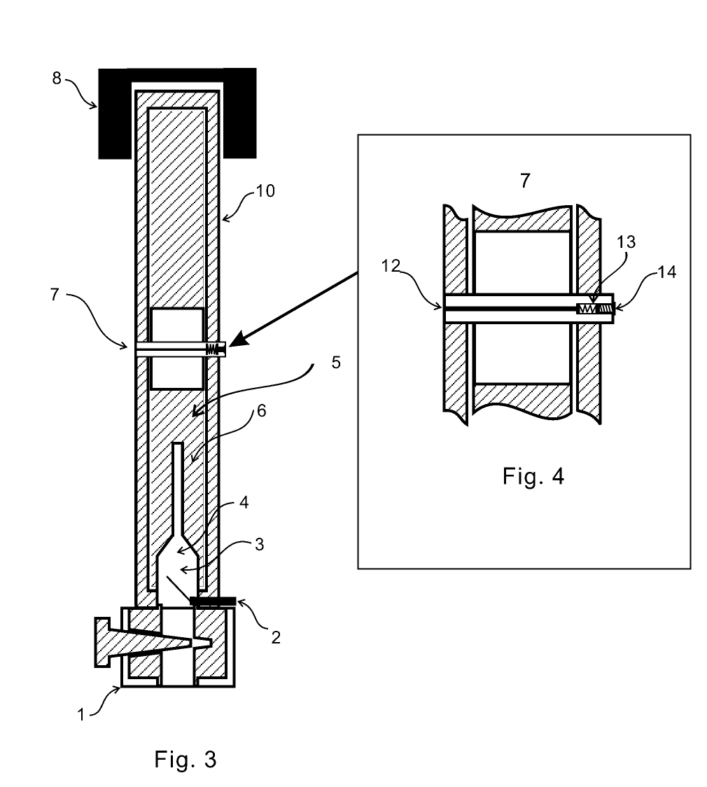

Gas enters into a wheeled chamber 10 [FIG. 2] with vessels 6 [FIGS. 2, 3]. The wheeled chamber has a diameter-width ratio of 10:1. The wheeled chamber is attached to a wheel axle system 7 [FIGS. 2, 3]. The wheel axle system is composed of an adjustable axle bolt 14 [FIG. 4] with a spring 13 [FIG. 4] attached to it which is set on a fixed axle 12 [FIG. 4]. The adjustable axle bolt can be rotated by user to tighten or loosen to make wheeled chamber more or less sensitive for gas entering the chamber.

The gas entering the vessels 6 [FIGS. 2, 3] of the wheeled chamber causes the wheel 5 [FIGS. 2,3] to rotate. A motion sensor 8 [FIGS. 2,3] recognizes the wheel rotation as a pulse signal and converts the pulse signal to a digital signal which is sent to the CPU 11 [FIG. 2] contained within the device. The CPU 11 [FIG. 2] calculates gas usage by a set of formulas in a built-in software program.

Gas then exits device from gas-out tube 9 [FIG. 2] and continues to delivery.

If mechanical valve is adjusted by user to disallow gas to pass into device, gas-in sensor 2 [FIGS. 2, 3] notifies CPU to stop calculating gas usage.

What is claimed is:

1. A flowmeter comprising: A gas chamber, the sensors installed in the gas chamber, and CPU connects to the sensor and LCD display device; the gas chamber assembly by shell, wheel, rotor sensor, gas-in sensor, cone shaped gas-in tube and gas-out tube; the function of the gas chamber is: when gas enters into the gas chamber, the pressure of gas causes the wheel to rotate and the rotor sensor can catch the rotation, and convert the signal to a digital signal, then send digital signal to CPU for data processing; a gas-in sensor connects to the gas chamber to sense the gas-in start signal and send the start signal to CPU; a wheel installed in the gas chamber by axle and the axle bolt can adjust the force of friction to set the wheel rotation speed; a gas valve installed with the gas-in tube in the gas chamber adjusts the flow of gas; a cone shaped gas-in tube increases the pressure of gas to make the wheel sensitive to rotation by gas pressure; the onboard programmable CPU is connected to the sensors, and receives the sensor's digital signal to process in certain modes programmed by CPU; a programmable display device such as LCD screen, connects to the CPU, and displays the information processed by CPU, which is the amount of gas used; for example, how much oxygen was passed from initial source such as a pressurized oxygen tank to a recipient, such as a patient's ventilation systems.
